(19) United States
(12) Patent Application Publication
(10) Pub. No.: US 2022/0233582 A1
(43) Pub. Date: Jul. 28, 2022

(54) Silicone Face Mask For Skin Treatment

(71) Applicant: Christine Marie Schrader, Parker, CO (US)

(72) Inventor: Christine Marie Schrader, Parker, CO (US)

(21) Appl. No.: 17/156,619

(22) Filed: Jan. 24, 2021

(51) Int. Cl. A61K 31/80

(52) U.S. Cl. CPC A61K 31/80

(57) ABSTRACT

An improved facial mask composed of non-adhesive medical grade silicone gel sheeting. The mask is configured to fit over a human face with openings for the eyes, nose and mouth, with a plurality of chevron cuts around the mask perimeter to aid contouring to the user's face, and an ear loop on each side of the mask to stabilize and securely hold the mask in place. The central portion of the mask is comprised of thicker silicone gel, extending from the area between the eyebrows, proceeding under the eyes, along each side of the nose, and circumventing the area around the mouth. The device is worn like any conventional mask. In this way, the non-adhesive medical grade silicone can provide direct and continuous treatment for the improvement of facial skin.

### CROSS-REFERENCE TO RELATED APPLICATIONS

Provisional application No. 62/965,317, filed on Jan. 24, 2020

### STATEMENT REGARDING FEDERALLY SPONSORED RESEARCH OR DEVELOPMENT

(not applicable)

### REFERENCE TO SEQUENCE LISTING, A TABLE, OR A COMPUTER PROGRAM LISTING COMPACT DISC APPENDIX

(not applicable)

### THE NAMES OF THE PARTIES TO A JOINT RESEARCH AGREEMENT

(not applicable)

### STATEMENT REGARDING PRIOR DISCLOSURES BY THE INVENTOR OR A JOINT INVENTOR

(not applicable)

### BACKGROUND OF THE INVENTION

Historically, medical grade silicone gel sheeting has been utilized as an effective treatment of epithelial wounds, burns, scars, and wrinkle mitigation. It is a soft, flexible, semi-occlusive product made from a dimethethylsiloxane cross linked polymer, reinforced with a silicone membrane backing. It is a proven and effective therapy due to its inhibiting of trans epidermal water loss, and it acts upon the process of refraining fibroblasts from producing excess collagen cells, thus softening epithelium and preventing scarring. Silicone gel sheeting treatment is most effective when it can be kept in maximum contact with the targeted areas, for a prolonged period of time.

Medical grade silicone gel sheeting is available in the form of adhesive sheets of various, pre-cut, rectangular sizes, or pre-shaped adhesive patches. The use of small, individual, silicone gel sheets or patches, fail to remain in place, especially during activity and sleep, and do not address the entire surface and contours of the facial areas.

The adhesive property added to the current industry's silicone gel sheeting, can provide discomfort to skin areas that are sensitive to its adhesives, especially if being used to advance the healing of facial incisions or lacerations, burn wounds, acne scarring, post-surgical facial procedures, post cosmetic facial procedures such as dermabrasion, micro needling and chemical peel procedures, or if the user has facial hair. In addition, pediatric patients may be especially sensitive and uncomfortable with the adhesive quality of current silicone gel sheet products. Additionally, facial masks for skin treatment, have not addressed the needs of the industry, due to their uniform thickness and limited ability to contour to facial areas.

### BRIEF SUMMARY OF THE INVENTION

The object of this invention is to provide a non-adhesive, medical grade silicone gel sheet mask, that fully covers and contours to the surface of the face, provided by variable mask thickness, and side ear loops. This invention allows maximum facial coverage that is secure, remains in place, and excludes the need for adhesives.

In view of the foregoing disadvantages inherent in the known types of facial silicone gel patches, the present invention provides a new silicone face mask wherein it can be worn as one complete unit and remain in place, even during sleep and activity.

The general purpose of the present invention, is to provide a new silicone gel sheet mask for wound, scar and wrinkle mitigation. The novel features of the present invention result in a new silicone gel face mask, which is not anticipated, rendered obvious, suggested, or even implied by the current industry addressing, either alone or in any combination thereof.

To attain this, the present invention provides a silicone gel face mask that covers the contours of the face (with the exception of the eyes, nostrils, and mouth areas), in one cohesive piece of silicone gel sheeting, thus overcoming the shortcomings of small silicone gel patches. The same mask has added thickness corresponding to the areas beginning between the eye brows, extending under the area below the eyes, then proceeding along the sides of the nose, and above and below the lip area. This feature of the innovation accommodates the anatomic features of the human face. It provides an anatomically adapted mask, due to the supplementary thickness of the mask in the areas just described, and due to this added structure of the product, it provides a secure, well-fitting mask that enhances its adherence and contact with the skin of the face.

Another stand out feature of the present invention is the side ear loops attached to the mask. These ear loops provide stability to the mask's secure placement and continuous contact with the surface of the face as well as eliminates the need for an adhesive additive to the silicone gel sheeting. Since the mask can remain in place due to the ear loop feature, the mask can be worn for lengthy periods of time, including during activity and sleep, where otherwise is not possible with silicone gel patches.

The present innovation is comprised of non-adhesive silicone gel sheeting. This permits wearers who have partial facial bandaging, sutures, fragile lacerations or wounds, sensitive skin, facial hair, and pediatric users, to wear, and remove the mask easily, and without discomfort.

### BRIEF DESCRIPTION OF THE DRAWINGS

Turning now descriptively to the drawings, this invention may be embodied in the form illustrated in the accompanying drawings, with attention being called to the fact that the drawings are illustrative only, and that changes may be made in the specific construction illustrated.

Page 1

FIG. 1 Is a front, forward view of the mask in accordance with the present invention.

FIG. 1.1 Demonstrates the areas of the mask that allows cut outs for the eyes.

FIG. 1.2 Demonstrates the area of the mask that allows a cut out for the nose.

FIG. 1.3 Demonstrates the area of the mask that allows a cut out for the mouth.

FIG. 1.4 Demonstrates the appearance and position of side ear loops.

Page 2

FIG. 2 Is a modified side perspective of the mask in accordance with the present invention.

FIG. 2.5 Demonstrates the areas of the mask that correspond to supplementary silicone gel thickness in those shaded area.

FIG. 2.6 Demonstrates where possible chevron cut outs in the mask may be positioned.

Page 3

FIG. 3 Is a side view of the mask in accordance with the present invention

### DETAILED DESCRIPTION OF THE INVENTION

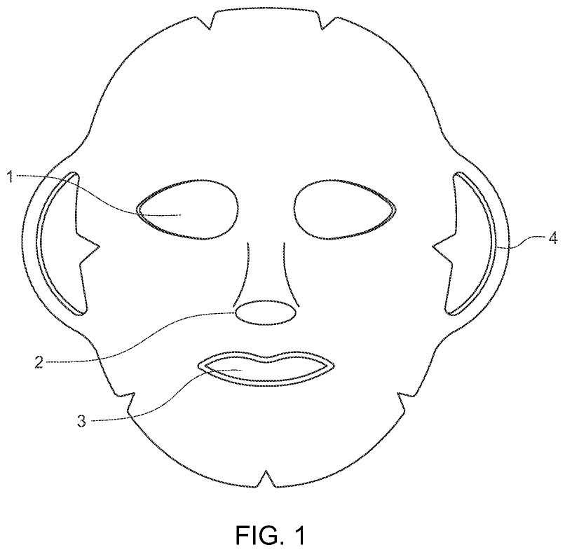

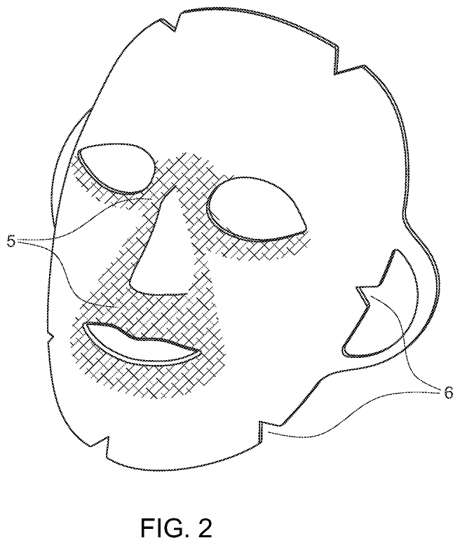

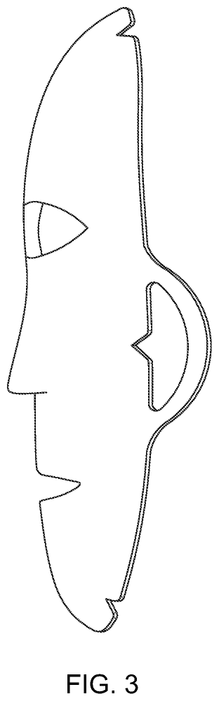

This is a mask composed of medical grade liquid silicone, which can be injection molded and cured to form a flexible mask that corresponds to the contours of the human facial area. The slightly convex, silicone gel mask, is designed to cover and fit over the frontal skin surface of a human the face. It has openings cut for the eyes (FIG. 1), the nose (FIG. 1.2), and the mouth (FIG. 1,3). The mask is in contact with the skin of the face, excluding the areas of the eyes, from just above the eyebrows, to below the eye's lower lashes, and circumventing the eye's inner canthus and outer canthus areas, as well as a cut opening for the nares area of the nose, and the right and left oral commissures of the lips. The mask has ear loops to assist keeping the mask in position (FIG. 1.4). The two ear loops extend from the left and right, median side margins of the mask. Each ear loop connects to the mask, just superior, and inferior, to the common position of an ear, one on the left side, and one on the right side of the mask. In addition, the mask has chevrons positioned around the margin of the mask (FIG. 2,6), to enhance its contact with the skin of the face. The mask has thicker silicone gel sheeting in specified areas of the mask that correlate to key facial areas, specifically designed to create consistent contact with the skin of the face, and to prevent gaps or buckling of the mask (FIG. 2.5). The thicker silicone gel begins at the glabella and extends along the entire left and right infraorbital margins. It then follows from the left and right supraorbital notches, down both sides of the nose, covering both the left and right nasal labial folds, then around the lips, encompassing the area of the left and right commissure of the lips, the upper lip cutaneous, and the mentolabial sulcas. The mask can be placed upon the face by holding it up, at approximately nose-level, then transferring the mask onto the surface of the face, and placing an ear loop over each ear to secure it. The mask can be removed in the same manner, by bringing each ear loop back over the ears, and allowing the mask to gently release from its contact with the skin of the face.

With respect to the above description, it is to be realized that the optimal dimensional relationships for the parts of the invention, to include variations in size, shape, form, manner of operation, assembly and use, and are deemed readily apparent and obvious to the one skilled in the art, and all equivalent relationships. Suitable modifications and equivalents may be resorted to, falling within the scope of invention.

1. I claim a face mask for skin treatment, the facial mask comprising:
a continuous sheet of non-adhesive medical grade silicone gel sheeting, formed to cover the entire frontal facial area, with cut outs for corresponding eyes, nose and mouth, and
an additional thickness of said non-adhesive medical grade silicone gel along the central areas of the mask beginning between the eyebrows and extending down along the sides of the nose and surrounding the mouth, and
an ear loop secured medially to each side edge of said face mask,
wherein the improvement provides non-adhesive medical grade silicone gel treatment to be placed in full, continuous contact, and held in secure position, to the entire facial area of the user.
